(19) United States
(12) Patent Application Publication
(10) Pub. No.: US 2008/0008679 A1
(43) Pub. Date: Jan. 10, 2008

(54) Osteoinduction-assisting composition

(75) Inventors: Tatsushi Kawai, Aichi (JP); Tomoo Suzuki, Aichi (JP); Mikito Deguchi, Kyoto (JP); Mitsuharu Mizuno, Kyoto (JP); Hidefumi Fujimura, Kyoto (JP)

Correspondence Address: STEVENS DAVIS MILLER & MOSHER, LLP, 1615 L STREET, NW, SUITE 850, WASHINGTON, DC, 20036, US

(73) Assignee: KABUSHIKI KAISHA SHOFU, Kyoto (JP)

(21) Appl. No.: 11/480,585

(22) Filed: Jul. 5, 2006

(51) Int. Cl. A61K 31/74; C08L 33/14

(52) U.S. Cl. 424/78.31; 525/223

(57) ABSTRACT

The present invention relates to a novel reactive medical substance having the bone restoration function, comprising a particular methacrylic acid ester, polymer and osteogenic factor. The reactive medical substance of the present invention has a mechanical strength which is increased by a reaction and is used in a living body for a long period of time. The osteoinduction-assistance composition having the bone restoration function of the present invention is characterized in that the composition maintains suitable elasticity, and can be easily formed with scissors or a sharp blade upon use. The reactive medical substance of the present invention comprises a mixture of a methacrylic acid ester, a polymer and a biological absorbing material, and can regenerate a mocosa, form a regenerated epithelium, and stabilize a transplanted piece.

### TECHNICAL FIELD

The present invention relates to a reactive medical substance having the bone restoring function for tissue regeneration excellent in operability and stability, which is used for inducing a tissue in the medical service field such as medical, dental and oral surgical fields.

### BACKGROUND ART

For re-constructing bone deficiency in the medical service region, a variety of transplantation materials have previously been applied. Generation of a bone needs a cell producing a bone, an osteogenic factor, and a scaffold for maintaining them. As the scaffold, autogeneic bones, allogenic bones, and biological materials are applied. These scaffolds have problems such that autogeneic bones have a problem of a restricted bone amount, and surgical invasion into a healthy tissue, allogeneis bones have a problem of infection and an immunological rejection reaction, and biological materials are unstable in biocompatibility and an osteoinduction amount.

In a scaffold for inducing a cell, metal materials, polymer materials, and ceramic materials have been applied. However, these materials have a problem in the ability to retain a growth factor in a living body, particularly, formation or morpholism impartation at operation.

As a desired nature for a scaffold for inducing a cell, it is required that a substance constituting a scaffold is easily adhered to a tissue, biocompatibility is better, and a rate of curing a bone deficient part is enhanced. In addition, it is required that a medical material has a strength in a body to some extent, and the material has adherability, morpholism imparting property and morpholism retainability at imbedding into a body, depending on an application site.

JP-A No. 2001-523999 discloses a polymerizable semipermeable network alloy for an orthopedic plate and a bone cement, which can be biologically deteriorated, and a process for producing this. However, this alloy does not exhibit morpholism imparting property, and morpholism retainability, and they can not be applied in the medical field in many cases. Furthermore, osteoinductive property is not sufficient.

An object of the present invention is to provide an osteoinduction-assisting composition having the bone restoration function, which improves biocompatibility which was insufficient in the previous material as a scaffold, has strong ability to retain a bone growth factor, promotes induction of a newborn bone, and improves adherability, morpholism imparting property and morpholism retainability which were a defect in the prior art, depending on an application site.

The present inventors found out that, upon preparation of a reactive regeneration medical substance having the bone restoration function, a reactive regeneration medical substance having the bone restoration function, which contains a compound represented by the chemical formula [1], and in which a kind and a blending ratio of a polymer are changed, is effective.

CH₂═C(CH₃)COO—(CH₂)ₙ—OCO—(CH₂)ₙ—COO—R [1]

The present invention is an osteoinduction-assisting composition having morpholism imparting property, and morpholism retainability which assists osteoinduction, and has a methacrylic acid ester, a polyfunctional polymerizable monomer, a polymer, a polymerization initiator, an osteogenic factor and a biocompatible substance.

The present invention is the osteoinduction-assisting composition according to claim 1, wherein the methacrylic acid ester is a methacrylic acid ester represented by the general formula [1] (wherein n represents an integer of 1 or more, and R represents a hydrogen atom or a methyl group).

The present invention is the osteoinduction-assisting composition according to claim 1, wherein the polymer comprises a polymer which can be dissolved or swollen in at least one of a methacrylic acid ester, and a polyfunctional polymerizable monomer, and a polymer which can not be dissolved or swollen in at least one of a methacrylic acid ester and a polyfunctional polymerizable monomer.

The present invention is the osteoinduction-assisting composition according to claim 1, wherein the osteogenic factor is a factor which has been extracted from a demineralized animal cortical bone and partially purified, or a factor which has been a molecular biologically artificially synthesized.

The present invention is the osteoinduction-assisting composition, wherein the biocompatible substance comprises a homopolymer or a copolymer of L-lactic acid, DL-lactic acid, glycolic acid, or ε-caprolactone.

The osteoinduction-assisting composition having the bone restoration function has better compatibility in a living body, is excellent in morpholism imparting property particularly at embedding into a living body and morpholism retainability after polymerization, and is extremely effective as a scaffold for inducing a bone.

Since bone production is rapid, curing progresses rapider than as usual, and recovery is also fast.

The osteoinduction-assisting composition having the bone restoration function of the present invention is characterized in that the composition maintains suitable elasticity, and can be easily formed with scissors or a sharp blade upon use.

### BRIEF DESCRIPTION OF THE DRAWING

FIG. 1 is a soft X-ray photograph of a transplanted part. In the figure, symbol 1 is a newborn bone induced part.

### BEST MODE FOR CARRYING OUT THE INVENTION

In the present invention, a representative compound represented by the chemical formula [1] is methyl methacryloyloxyethylsuccinate, (hereinafter, abbreviated as TA in some cases), methyl methacryloyloxyethylglutarate, and methyl methacryloyloxyethyladipate. The compound is preferably TA, or methyl methacryloyloxyethylglutarate, further preferably TA. A preferable blending amount is 10.0 to 90.0% by mass, preferably 10.0 to 75.0% by mass.

TA is the most preferable compound for implementing the present invention.

The polyfunctional polymerizable monomer is a methacrylic acid ester compound represented by the chemical formula [1], and a polyfunctional polymerizable monomer which is copolymerizable therewith. Examples of such the polyfunctional polymerizable monomer include polymerizable monomers which are generally used as a dental material such as polyfunctional monomers having at least two ethylenic double bonds, ethylene glycol di(meth)acrylate, triethylene glycol di(meth)acrylate, butylene glycol di(meth)acrylate, trimethylolpropane trimethacrylate, 2,2-bis[4-phenyl]proprane di(meth)acrylate, dimethacrylic acid 1,6-hexanediol (hereinafter, abbreviated as “HX”), UDMA, and Bis-GMA. The polyfunctional polymerizable monomer is preferably HX.

A preferable blending amount is 5.0 to 50.0% by mass, preferably 10.0 to 40.0% by mass.

The polymer which can be dissolved or swollen in at least one of a methacrylic acid ester and a polyfunctional polymerizable monomer is a polymer or a copolymer of alkyl (meth)acrylate, preferably a homopolymer or a copolymer obtained by using, as a main component, MMA, ethyl methacrylate butyl methacrylate, or propyl methacrylate. The polymer is preferably a homopolymer or a copolymer of polymethyl methacrylate, polyethyl methacrylate, polybutyl methacrylate or polypropyl methacrylate.

A preferable blending amount of the polymer which can be dissolved or swollen in at lest one of a methacrylic acid ester and a polyfunctional polymerizable monomer is 20.0 to 75.0% by mass, more preferably 30.0 to 65.0 by mass.

The polymer which can not be dissolved or swollen in at least one of a methacrylic acid ester and a polyfunctional polymerizable monomer is most preferably carboxymethylcellulose (hereinafter, abbreviated as CMC), sodium alginate (hereinafter, abbreviated as ANa), xanthan gum, cellulose derivatives, saccharides or polysaccharides. The polymer is preferably saccharides or polysaccharides.

A preferable blending amount of the polymer which can not be dissolved or swollen in at least one of a methacrylate acid ester and a polyfunctional polymerizable monomer is preferably 0.5 to 20.0% by mass, more preferably 1.0 to 15.0% by mass.

The polymer is a powder having an average particle diameter of preferably 4 to 500 μm, more preferably 1 to 85 μm. A molecular weight of an alkyl (meth)acrylate polymer is most preferably 100 thousands to 1 million.

As an osteogenic factor, an osteogenic factor which was extracted from a demineralized bovine cortical bone and partially purified according to the method of Urist et al. and for which physiologically activity was confirmed by in vivo transplantation is optimal in both of quantity and quality. An osteogenic factor obtained by extracting from an animal species other than a cattle and teeth, and purifying this has no problem. Alternatively, an osteogenic factor which was molecular biologically artificially synthesized may be applied. Further, demineralized substrates themselves may be used. A preferable blending amount of an osteogenic factor is 0.5 to 30.0% by mass, preferably 1.5 to 25.0% by mass.

It is preferable that the biocompatible substance comprises a homopolymer or a copolymer of L-lactic acid, DL-lactic acid, glycolic acid, or ε-caprolactone.

A preferable blending amount of the biocompatible substance is 0.5 to 45.0% by mass, preferably 1.5 to 35.0% by mass.

The polymerizable initiator in the present invention can be arbitrarily selected depending on a polymerization form which is suitable for the purpose. In order to polymerize a reactive medical substance having the bone restoration function, a range of 60° C. or lower is better and, thereupon, as a polymerization initiator, peroxide and AIBN are effective. When polymerized with ultraviolet-ray or visible light, a photopolymerization initiator and a reducing agent are added at 0.2 to 0.5 by weight relative to a mixture of a methacrylic acid ester and a polyfunctional polymerizable monomer. Specifically, as a photopolymerization initiator, α-diketone compound, a ketal compound and an anthraquinone-based compound are effective and, particularly, camphorquinone (hereinafter, abbreviated as “CQ”) of an α-diketone compound is preferable. As a reducing agent, primary, secondary or tertiary amine is effective, and metacrylic acid dimethyl ester of tertiary amine is particularly preferable. Alternatively, dibutyltin dilaurate (hereinafter, abbreviated as “Bu-Tin”) compound is preferable.

As a process for preparing a reactive medical substance having the bone restoration function, an osteoinduction-assisting composition (mixture) of a polymer, TA, a polyfunctional polymerizable monomer, an osteogenic factor, a biocompatible substance and a polymerization initiator is filled into a mold or a gypsum mold for preparing a reactive medical substance having the bone restoration function, this is, pre-pressed at 20 to 500 Kgf/cm² for about 10 to 120 minutes, and a product is taken out from the mold and is subjected to form correction. A reactive medical substance having the restoration function is stored at room temperature or lower, more preferably in a simple closed container at 5° C. to 25° C. Particularly, it is necessary that a photopolymerization type is shielded from light in order to inevitably avoid ultraviolet-ray and visible light.

### EXAMPLES

The present invention will be explained specifically below by Examples and Comparative Examples.

Among cell induction factors, an osteogenic factors, and Bone Morphogenic Protein (BMP) were prepared using a pig cortical bone as a starting material according to the method of Urist et. al. That is, a cortical bone was demineralized with 6 M hydrochloric acid, and the demineralized bone substrate was continuously treated with EDTA and calcium chloride. This bone substrate was solubilized using a protein dissolving agent such as guanidine hydrochloride and urea, and it was subjected to desalting treatment with a semipermeable membrane using distilled water as a dialysis external solution to recover a non-water-soluble fraction. This fraction was dissolved in a protein dissolving agent again, this was subjected to citric acid dialysis and water dialysis to remove polymer components, liquid components were removed with an organic solvent, and this was dried to prepare a partially purified osteogenic factor. A purity can be further increased by liquid chromatography. In order to investigate physiological activity, about 3 mg of the resulting osteogenic factor was transplanted into a space between femoral muscles of a 4 week old male mouse, and 3 weeks after transplantation, induction of a newborn bone was confirmed from a soft X-ray photographic image and a pathological tissue image. A newborn bone having a size of about 1 square mm was usually induced per 1 mg of an osteogenic factor on a soft X-ray photographic image.

### Example 1

A mixed solution containing 3.6 g of TA, 2.4 g of HX, 0.036 g of CQ and 0.18 g of a Bu-Tin compound, and 10 g of PEMA-1 (molecular weight 250 thousands, average particle diameter 25 micron PEMA) were mixed. A method of mixing a mixed solution and PEMA-1 can be performed by a method such as 1) mortar mixing, 2) container mixing, and 3) ball mill mixing, and, this time, the mixing method was performed using “Experimental Planet Ball Mill, P-5” manufactured by Fritsch Japan Co., Ltd.). The mixing condition was under room temperature, a rotation number of 110/min, a mixing time of 10 min, and the number of balls amount 5 (diameter 10 mn).

After the polymer was swollen in a mixed solution, a pressure was applied at 20 to 80 kgf/cm² for 10 to 25 minutes in a mold (diameter 60 mm, thickness 0.5 mm), to prepare a reactive medical substance having the bone restoration function. Forming property, and a hardness and bending property after polymerization of a reactive medical substance having the bone restoration function are shown in Table 1. Morpholism formation with scissors was better and morpholism maintenance was stable. The reactive medical substance having the bone restoration function which had been photopolymerized with a visible light polymerization instrument, “Solidlite” (manufactured by SHOFU) for 3 minutes exhibited better polymerizability. Results are shown in Table 1.

### Example 2

A mixed solution containing 3.6 g of TA, 2.4 g of HX, 0.036 g of CQ and 0.185 g of a Bu-Tin compound, and a mixture containing 9.0 g of PEMA-1 and 0.1 g of DL polylactic acid “PLA-0020” (manufactured by Wako Pure Chemical Industries, Ltd.) were mixed. According to the same manner as that of Example 1 except that polylactic acid was used, a reactive medical substance having the bone restoration function was prepared and assessed. Morpholism formation with scissors was better and morpholism maintenance was stable. The substance having the bone restoration function which had been photopolymerized with a visible light polymerization instrument “Solidlite” manufactured by SHOFU) for 3 minutes exhibited better polymerizability. Results are shown in Table 1.

### Example 3

A mixed solution containing 3.6 g of TA, 2.4 g of HX, 0.036 g of CQ and 0.18 g of a Bu-Tin compound, 8.0 g of PEMA-1 and 2.0 g of DL polylactic acid “PLA-0020” (manufactured by Wako Pure Chemical Industries, Ltd.) were mixed. According to the same manner as that of Example 1 except that polylactic acid was used, a reactive medical substance having the bone restoration function was prepared, and assessed. Morpholism formation with scissors was better, and morpholism maintenance was stable. The reactive medical substance having the bone restoration function which had been photopolymerized with a visible light polymerization instrument “Solidilite” (manufactured by Shofu) for 3 minutes exhibit better polymerizablity. Results are shown in Table 1.

### Example 4

A mixed solution containing 3.06 g of TA, 2.4 g of HX, 0.036 g of CQ and 0.18 g of a Bu-Tin compound, and 7.0 g of PEMA-1 and 3.0 g of DL polylactic acid “PLA-0020” (manufactured by Wako Pure Chemical Industries, Ltd.) were mixed. According to the same manner as that of Example 1 except that polylactic acid was used, a reactive medical substance having the bone restoration function was prepared, and assessed. Morpholism formation with scissors was better, and morpholism maintenance was stable. The reactive medical substance having the bone restoration function which had been photopolymerized with a visible light polymerization instrument “Solidilite” (manufactured by Shofu) for 3 minutes exhibited better polymerizablity. Results are shown in Table 1.

### Example 5

A mixed solution containing 3.06 g of TA, 2.4 g of HX, 0.036 g of CQ and 0.18 g of a Bu-Tin compound, and 5.0 g of PEMA-1 and 5.0 g of DL polylactic acid “PLA-0020” (manufactured by Wako Pure Chemical Industries, Ltd.) were mixed. According to the same manner as that of Example 1 except that polylactic acid was used, a reactive medical substance having the bone restoration function was prepared, and assessed. Morpholism formation with scissors was better, and morpholism maintenance was stable. The reactive medical substance having the bone restoration function which had been photopolymerized with a visible light polymerization instrument “Solidilite” (manufactured by Shofu) for 3 minutes exhibited better polymerizablity. Results are shown in Table 1.

### Example 6

A mixed solution containing 3.06 g of TA, 2.4 g of HX, 0.036 g of CQ and 0.18 g of a Bu-Tin compound, and 8.0 g of PEMA-1 and 2.0 g of DL polylactic acid “PLA-0020” (manufactured by Wako Pure Chemical Industries, Ltd.) were mixed. According to the same manner as that of Example 1 except that polylactic acid was used, a reactive medical substance having the bone restoration function was prepared, and assessed. Morpholism formation with scissors was better, and morpholism maintenance was stable. The reactive medical substance having the bone restoration function which had been photopolymerized with a visible light polymerization instrument “Solidilite” (manufactured by Shofu) for 3 minutes exhibited better polymerizability. Results are shown in Table 1.

### Example 7

A mixed solution containing 3.6 g of TA, 2.4 g of HX, 0.036 g of CQ and 0.18 g of a Bu-Tin compound, 8.0 g of PEMA-1 2.0 g of ANa were mixed. According to the same manner as that of Example 1 except that ANa was used, a reactive medical substance having the bone restoration function was prepared, and assessed. Morpholism formation with scissors was better, and morpholism maintenance was stable. The reactive medical substance having the bone restoration function which had been photopolymerized with a visible light polymerization instrument “Solidilite” (manufactured by Shofu) for 3 minutes exhibited better polymerizability. Results are shown in Table 1.

### Comparative Example

Using a regeneration medical membrane “DEXON MESH” (manufactured by DAVIS & GECK), formation with scissors and photopolymerizability were assessed as in Examples 2 to 7. Formation with scissors had no elasticity, morpholism could not be retained, and there was no photopolymerizability. Results are shown in Table 1. A numerical value in a parenthesis is a standard deviation.

TABLE 1

| | | | Bending property | | | | | |
|---|---|---|---|---|---|---|---|---|
| | Formation with | | Bending maximum stress (MPa) | | | Young's modulus (GPa) | | |
| | scissors and shape | | After 1 | After 7 | After 14 | After 1 | After 7 | After 14 |
| | maintenance | Polymerizability | day | days | days | day | days | days |
| Example 1 | Formation is better, | Better | 54.6(2.5) | 60.5(2.2) | 61.1(0.7) | 1.27(0.06) | 1.39(0.03) | 1.37(0.03) |
| | Shape maintenance is | | | | | | | |
| | better. | | | | | | | |
| Example 2 | Formation is better, | Better | 36.0(4.9) | 35.3(6.3) | 38.3(6.1) | 1.08(0.06) | 1.35(0.04) | 1.29(0.06) |
| | Shape maintenance is | | | | | | | |
| | better. | | | | | | | |
| Example 3 | Formation is better, | Better | 23.6(2.9) | 23.1(3.0) | 18.8(1.0) | 0.93(0.03) | 1.17(0.04) | 1.09(0.11) |
| | Shape maintenance is | | | | | | | |
| | better. | | | | | | | |
| Example 4 | Formation is better, | Better | 20.1(4.0) | 15.7(2.3) | 14.4(1.6) | 0.79(0.13) | 1.02(0.04) | 0.83(0.12) |
| | Shape maintenance is | | | | | | | |
| | better. | | | | | | | |
| Example 5 | Formation is better, | Better | 16.0(2.9) | 12.1(0.5) | 12.0(0.3) | 0.38(0.16) | 0.81(0.04) | 0.60(0.05) |
| | Shape maintenance is | | | | | | | |
| | better. | | | | | | | |
| Example 6 | Formation is better, | Better | 23.0(4.5) | 23.2(3.6) | 20.2(2.4) | 0.97(0.03) | 1.17(0.04) | 1.09(0.11) |
| | Shape maintenance is | | | | | | | |
| | better. | | | | | | | |
| Example 7 | Formation is better, | Better | 25.0(3.5) | 24.5(3.0) | 22.4(3.2) | 1.06(0.05) | 1.38(0.04) | 1.19(0.11) |
| | Shape maintenance is | | | | | | | |
| | better. | | | | | | | |
| Compar- | Shape maintenance | Not | Unmeas- | Unmeasurable | Unmeasurable | Unmeasurable | Unmeasurable | Unmeasurable |
| ative | is impossible. | polymerized | urable | | | | | |
| Example | | | | | | | | |

### [Method of Assessing Polymerization Molded Article]

Assessment of physical properties of cured articles obtained by polymerization and molding in Examples and Comparative Example were performed according to the following method.

### Bending Strength

A sample having a test piece size of a width (2 mm), a thickness (2 mm) and a length (25 mm) was prepared, this was stored in water at 50° C. for 24 hours, 7 days and 14 days, and bending properties (maximum stress, Young's modulus) were measured using “Autograph AG5000B” (manufactured by Shimadzu Corporation). The number of test pieces was 5. Measuring condition was a distance between supports of 20 mm, and a crosshead speed of 1 mm/min.

### Example 8

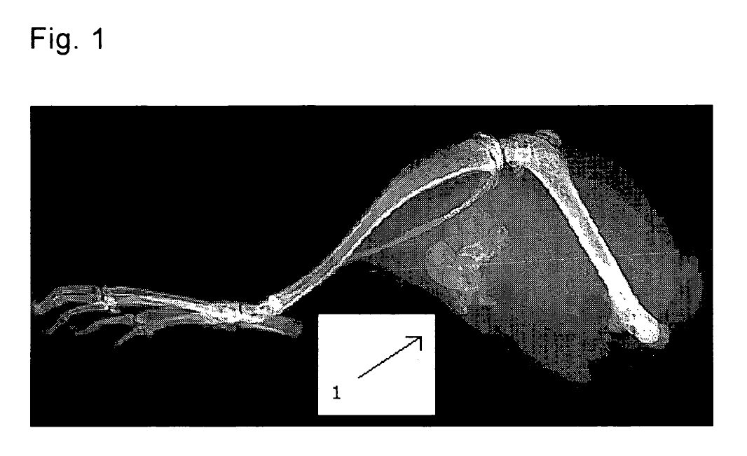

3 mg of BMP was uniformly dispersed on a surface (longitudinal 8 mm, transverse 5 mm) of the reactive medical substance having the bone restoration function prepared in Example 2, this was transplanted into a space of a femoral fascia of a 4-week old mouse and, 4 weeks after transplantation, the mouse was slaughtered with diethyl ether, and image diagnosis and histological observation with a soft X-ray photograph of a transplanted part were performed. In addition, biocompatibility was assessed by the presence or the absence of encapsulation with a surrounding fibrous connective tissue, and an inflammatory cell. In a soft X-ray photograph tissue image, a newborn bone was induced along a shape of a reactive medical substance having the bone restoration function. Biocompatibility was better. Results are shown in Table 2. Image diagnosis of a newborn bone with a soft X-ray photograph of a transplanted part is shown in FIG. 1.

### Examples 9 to 13

3 mg of BMP was uniformly dispersed on a surface (longitudinal 8 mm, transverse 5 mm) of the reactive medical substance having the bone restoration function prepared in Example 3, this was transplanted into a space of a femoral fascia of a 4-week old mouse and, 4 weeks after transplantation, the mouse was slaughtered with diethyl ether, and image diagnosis and histological observation with a soft X-ray photograph of a transplanted part were performed. In addition, biocompatibility was assessed by the presence or the absence of encapsulation with a surrounding with a fibrous connective tissue, and an inflammatory cell. In a soft X-ray photograph tissue image, a newborn bone was induced along a shape of a reactive medical substance having the bone restoration function. Biocompatibility was better. Results are shown in Table 2.

TABLE 2

| | | Induction of | |
|---|---|---|---|
| | | newborn bone at | Biocompatibility |
| | BMP | space of mouse | with fibrous |
| | amount (mg) | femoral fascia | connection tissue |
| Example 8 | 1.0 | Presence of | Presence of |
| | | induction of | biocompatibility |
| | | newborn bone | |
| Example 9 | 1.0 | Presence of | Presence of |
| | | induction of | biocompatibility |
| | | newborn bone | |
| Example 10 | 1.0 | Presence of | Presence of |
| | | induction of | biocompatibility |
| | | newborn bone | |
| Example 11 | 1.0 | Presence of | Presence of |
| | | induction of | biocompatibility |
| | | newborn bone | |
| Example 12 | 1.2 | Presence of | Presence of |
| | | induction of | biocompatibility |
| | | newborn bone | |
| Example 13 | 1.2 | Presence of | Presence of |
| | | induction of | biocompatibility |
| | | newborn bone | |

### (Preparation of BMP)

Based on the Urist method, extraction and purification were performed. That is, using a bovine cortical bone as a starting raw material, a soft tissue was removed, a bone groove tissue was ground and demineralized, this was extracted using a protein solubilizer and, thereafter, particularly polymer components were removed by multi-stage treatment, and BMT (osteogenic factor) having around 10 KDa to 40 kDa was extracted and purified.

### (Extraction of BMT)

A fresh cannon bone of a material cattle for preparing bovine cortical bone-derived partially purified BMP was obtained from a slaughterhouse, and BMP was extracted. Extraction was performed by 3 steps of demineralization, neutralization and protein extraction.

### (Process of BMP Extraction)

(1) Obtaining of fresh bone; Within 1 to 2 hours after slaughter for obtaining a fresh bone, a bovine lower limb bone was obtained from a slaughterhouse. Since whether a fresh bone can be obtained or not is greatly influences on activity of BMP, a dealer was made to understand importance of obtaining of a fresh bone in advance, and a dealer having a shortest transporting time was selected. Since in a season having a high air temperature, activity of an extracted protein is remarkably reduced due to action of an endgeneous BMP degrading enzyme, an extraction procedure was performed mainly in a term of November to April and an attention was paid to refrigeration and storage as much as possible so as not to raise a temperature of a bovine lower limb bone also in winter. (2) Removal of bovine skin and soft tissue; A joint part was first cut with a surgical knife, a hoof was severed and a bovine skin and a meat tissue of a bovine lower limb bone were removed. (3) Removal of epiphysis; Both ends of a lower limb bone with only a remaining periosteum were cut with an electric circular saw. (4) Removal of periosteum; A bone was grasped firm with gloves preventing sliding such as cotton work gloves, the bone was notched with a surgical knife into a strip in a longitudinal direction, and a periosteum was peeled off. (5) Removal of bone marrow tissue; A bone marrow was removed with a relatively hard tooth brush, and washed with water. (6) Grinding a bovine bone; Liquid nitrogen was placed into a relatively large expanded styrene, and a bovine bone remaining as only a cortical bone part was placed therein and frozen. This was ground into a size of around 1 mm with a grinder. (7) Defatting of ground bone; A ground bone was immediately placed into about 40 L of a chloroform methanol 1:1 solution, and this was stirred sufficiently for 4 to 5 hours. A solution was exchanged once during stirring. (8) Drying of ground defatted bone; A solution of a ground defatted bone in dry chloroform methanol was sufficiently squeezed with a gauze, and the ground defatted bone was spread on a new gauze having a size of a bed sheet, and dried overnight.

### (Demineralization and Neutralization of BMP)

Demineralization; A ground defatted dry bone was placed into 50 L of 0.6 N hydrochloric acid, and this was demineralized in a refrigerator at 4° C. for 3 days while stirring. Hydrochloric acid was exchanged with fresh one twice a day. Since when hydrochloric acid is vaporized, it damages refrigerator instruments, a demineralization tank should have a closed structure. After completion of demineralization, stirring and washing with distilled water were performed for 1 hour. This is continuous treatment, particularly a step proposed by Dr. Urist. Since treatment is troublesome and theoretical support for the effect is not clear, this stage is omitted and a next procedure is performed in some cases. However, the ability to induce a bone of partially purified BMP is elevated more in the case of treatment. Since a pH of a demineralized bone is finally increased to around neutral, treatment of considered to be effective. After completion of lyophilization and continuous treatment, a demineralized bone was washed well with distilled water, and hydro-extraction was performed using a gauze. After freezing at −80° C., lyophilization was performed.

### (Extraction of BMP)

Extraction of bone protein; A demineralized and lyophilized bone was immersed in 30 L of a 6M urea and 2M calcium chloride solution (containing an enzyme inhibitor), and this was mildly stirred for 24 hours to extract an intraosseous protein. It should be noted that although addition of 2 N calcium chloride remarkably increases extraction of protein in a mineralized bone substrate, a defective reagent is mixed in even a guranteed reagent in some cases as in urea.

### (Procedure of Purification of BMP)

Purification of the BMP extract was performed via steps of concentration, dialysis, centrifugation, citric acid dialysis, defatting, and lyophilization.

### (Concentration)

Concentration of extract; A large size ultrafiltration machine was used for concentrating an extract. The extract was filtered with a gauze, and suction-filtered using a filter paper. When solid components are mixed in, a filter of an ultrafiltration machine is clogged, and not only an extraction efficiency is reduced, but also a fitting part of a pump is fatally damaged in some cases. It takes about 6 hours to concentrate 30 L to around 2 L and, during this time, tap water was introduced into a jacket for cooling a pump and a solution reservoir was cooled with an ice so as not to raise a temperature of a solution.

### (Dialysis)

Desalting was performed using a membrane with a dialysis cutoff of 5000. In a chamber at a low temperature of 4° C., dialysis was performed for 72 hours and distilled water was exchanged with fresh one every 12 hours. Since when 6 M urea is used, an interior of a dialysis tube is coagulated into a gel, coagulation was dissolved with warm water every dialyzate exchange and, therefore, dialysis was performed.

### (Centrifugation)

Since BMP was present in a non-water-soluble part, a sediment was recovered with a large size centrifugal machine. A gel was dissolved before centrifugation, and first centrifugation was performed under 37° C./5000 g/20 min. Second and third centrifugations were performed under 4° C./5000 g/20 min after precipitates were suspended in distilled water.

### (Citric Acid Dialysis)

The recovered sediment was dissolved in 3 L of an extracting solution again, undissolved components were removed by centrifugation, and dialysis was performed for 24 hours using, as an external dialyzate, a 0.25 M sodium citrate buffer (pH 3.1) at a 11-fold amount that of a dialyzate in a dialysis membrane. Centrifugation was performed under 37° C./5000 g/20 min. Generated precipitates were recovered, second and third centrifugations were performed under 4° C./5000 g/20 min, and precipitates were washed.

### (Defatting)

A centrifuge tube was inverted to sufficiently perform hydro-extraction, and this was suspended in 2 L of a chloroform methanol 1:1 solution, thereby, defatting was performed. Unless not only defatting and removal of water at this stage are sufficiently performed, the recovered substance turns black. After defatting for 3 hours, partially purified BMP was recovered on a filter paper by suction filtration. The BMP was stored in a suction desiccator, and suction-dried with an aspirator overnight. After completion of dialysis, an external dialyzate was exchanged with a sufficient amount of distilled water and dialysis was further performed for 3 days. Distilled water was exchanged twice a day.

### (Lyophilization)

Centrifugation was performed 3 times under 4° C./500 g/20 min. A sediment was recovered and lyophilized. Since a series of treatments before a first defatting procedure extremely undergo influence of a temperature, it is important to complete treatment at once under a low temperature environment after a fresh bone is obtained. In later treatments, inactivation occurs during a process of dialysis in many cases and, therefore, it is necessary to perform procedures under as clean as possible environment so as to prevent mixing in of various bacteria.

### (Test of Activity of Heterotopic Osteogenesis Ability)

In order to investigate the newborn bone inducing ability of BMP used in experiment, each 5 mg of BMP was weighed, encapsulated in a gelatin capsule (Japanese Pharmacopoeia #5) and lyophilized. This sample was transplanted into a left femoral interfascial space of a 4-week old male ddY conventional mouse. Three weeks after transplantation, the mouse was slaughtered under diethyl ether anesthesia, and a soft X-ray photograph of a transplanted part was taken (30 kV, 4 mA, 40 sec). An impermeable structure exhibiting a bone beam structure was observed in a transplanted site. Further, in histopathological observation, a bone tissue, a cartilage tissue and a bone marrow tissue were observed. BMP was transplanted into a mouse femoral soft tissue and, 3 weeks after, a soft X-ray photograph of the same side was taken and a X-ray impermeable image was confirmed around a transplanted part. In addition, a pathological tissue strip around a transplanted part was prepared, stained with HE and this was observed under a light microscope. A newbone tissue was confirmed in a weakly magnified image and a strongly magnified image. In the presence of BMP, this was confirmed on SDS-PAGE. The presence of BMP was confirmed around 18 kDa and 38 kDa.

Thus, by encapsulating BMP in a gelatin capsule to enhance local retention, the activity was clearly confirmed.

What is claimed is:

1. An osteoinduction-assisting composition for assisting osteoinduction having morpholism imparting property and morpholism retainability, comprising a methacrylic acid ester, a polyfunctional polymerizable monomer, a polymer which can be dissolved or swollen in at least one of the methacrylic acid ester and the polyfunctional polymerizable monomer, a polymerization initiator and an osteogenic factor.

2. The osteoinduction-assisting composition according to claim 1, wherein the methacrylic acid ester is a methacrylic acid ester represented by the following general formula [1]:
CH₂═C(CH₃)COO—(CH₂)_{N}—OCO—(CH₂)ₙ—COO—R [1]
(wherein n represents an integer of 1 or more, and R represents a hydrogen atom or a methyl group).

3. The osteoinduction-assisting composition according to claim 1, wherein the polymer which can not be dissolved or swollen in at least one of the methacrylic acid ester and the polyfunctional polymerizable monomer is blended in the osteoinduction-assisting composition.

4. The osteoinduction-assisting composition according to claim 1, wherein the osteogenic factor is a substance obtained by extracting from a demineralized animal cortical bone and partially purifying it, or a factor which was molecular biologically artificially synthesized.

5. The osteoinduction-assisting composition according to claim 1, wherein one or more kinds copolymers selected from the group consisting of gelatin, hyaluronic acid, starch, polylactic acid, polyglycolic acid, and a mixture of lactic acid and glycolic acid which are biocompatible substances are blended in the osteoinduction-assisting composition.
